(19) United States
(12) Patent Application Publication
(10) Pub. No.: US 2026/0216433 A1
(43) Pub. Date: Jul. 30, 2026

(54) WEARABLE AUTO-INJECTION SYSTEMS AND METHODS

(71) Applicant: Purdue Research Foundation, West Lafayette, IN (US)

(72) Inventor: Muhammad Mustafa Hussain, West Lafayette, IN (US)

(73) Assignee: Purdue Research Foundation, West Lafayette, IN (US)

(21) Appl. No.: 19/454,104

(22) Filed: Jan. 20, 2026

Related U.S. Application Data
(60) Provisional application No. 63/749,372, filed on Jan. 24, 2025.

(51) Int. Cl. A61M 5/20

(52) U.S. Cl. CPC A61M 5/20; A61M 2005/206; A61M 2202/04; A61M 2205/18; A61M 2205/273; A61M 2205/3303; A61M 2205/70

(57) ABSTRACT

A wearable autonomous anaphylaxis treatment device for preventing life-threatening allergic reactions includes integrated physiological monitoring, artificial intelligence-based detection, and automated epinephrine injection without requiring manual user intervention. The device continuously monitors respiratory function, cardiovascular performance, and autonomic responses through multiple sensors while a processor analyzes sensor data to establish personalized baseline values and detect deviations. The processor identifies anaphylaxis through multi-system pattern recognition requiring concurrent respiratory distress and cardiovascular shock conditions combined with confidence scoring that distinguishes true anaphylactic episodes from false positive situations including anxiety, exercise, or isolated physiological variations. A spring-actuated injection mechanism delivers intramuscular epinephrine when detection criteria are satisfied and contact sensors confirm proper device placement. Safety features include alert-before-inject notification with user cancellation option and single-dose lockout preventing repeated actuation. The layered architecture integrates sensors, electronics, drug delivery, and power management in a compact wearable form factor suitable for continuous protection during daily activities.

### CROSS-REFERENCE TO RELATED APPLICATIONS

The present application claims priority to U.S. Provisional Application No. 63/749,372 filed Jan. 24, 2025, titled “WEARABLE AUTO-INJECTION SYSTEMS AND METHODS,” which is hereby incorporated by reference in its entirety.

### TECHNICAL FIELD

The embodiments disclosed herein generally relate to wearable medical devices for autonomous health monitoring and emergency intervention, and more specifically to wearable autonomous anaphylaxis treatment systems with integrated multi-modal physiological sensing, artificial intelligence-based diagnostic algorithms, and automated intramuscular drug delivery mechanisms.

### BACKGROUND

Anaphylaxis represents a serious life-threatening allergic reaction affecting millions of individuals annually. When exposed to allergens, affected individuals can experience anaphylaxis within seconds to minutes, triggering immune system responses that cause blood pressure drops, airway narrowing, breathing obstruction, skin reactions, and irregular heart rate patterns. Statistical analysis indicates that one in every twenty Americans experiences anaphylaxis, with fifty percent of adults and forty percent of children with food allergies having experienced severe reactions. Emergency department presentations and hospitalizations for anaphylaxis result in fatalities in approximately one percent of cases, contributing to substantial annual healthcare costs.

Current anaphylaxis treatment relies primarily on manual epinephrine auto-injectors requiring user-initiated administration. These conventional devices present limitations including delayed treatment due to recognition challenges, unavailability during critical moments, lack of continuous monitoring capabilities, and dependence on manual actuation by individuals experiencing severe physiological distress. Studies demonstrate that one-third of patients fail to use available epinephrine auto-injectors even when accessible, often due to uncertainty about symptom severity or hesitation during emergency situations.

Existing wearable health monitoring devices provide physiological parameter tracking but lack integrated therapeutic intervention capabilities. Smart wearable technologies monitor vital signs including heart rate and activity levels, yet these systems cannot autonomously detect anaphylaxis patterns or deliver emergency medication. Conversely, conventional auto-injectors provide drug delivery functionality but operate entirely through manual actuation without integrated symptom monitoring or diagnostic capabilities.

Recent development efforts have explored wearable epinephrine delivery concepts, though technical challenges remain unresolved. Some approaches redesign conventional auto-injectors into wearable configurations while maintaining manual actuation requirements. Other initiatives investigate invasive blood sampling techniques for histamine detection, introducing complexity associated with continuous blood access, sample processing requirements, and delayed detection timeframes. These conventional approaches continue to separate monitoring functions from intervention capabilities, requiring users to recognize symptoms and manually initiate treatment during physiological emergencies.

The fundamental challenges of anaphylaxis management include accurate real-time symptom recognition, differentiation from similar conditions presenting overlapping symptoms, immediate intervention during critical response windows, and autonomous operation when individuals cannot self-administer treatment. Current technologies fail to address these challenges through integrated solutions combining continuous physiological monitoring, intelligent pattern recognition, and automated therapeutic delivery in wearable form factors suitable for daily use.

Consequently, there exists a need for improved autonomous medical intervention systems that continuously monitor multiple physiological parameters, accurately detect anaphylaxis through multi-system pattern analysis, distinguish true anaphylactic episodes from false positive conditions, and autonomously deliver intramuscular epinephrine without requiring manual user intervention. Accordingly, various embodiments detailed herein provide improved systems and methods for wearable autonomous anaphylaxis detection and treatment.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

In one aspect, variations of the disclosed wearable autonomous anaphylaxis treatment device may include integrated physiological monitoring components, intelligent diagnostic processing systems, and automated intramuscular injection mechanisms. The device comprises multiple physiological sensors configured to continuously monitor respiratory function, cardiovascular performance, and autonomic responses, generating comprehensive sensor data indicative of physiological conditions. A processor analyzes the sensor data to establish personalized baseline physiological states, detect deviations from baseline parameters, identify multi-system conditions including respiratory distress and cardiovascular shock, generate confidence scores based on condition presence, and trigger automated intervention when predetermined thresholds are exceeded. An injection mechanism delivers therapeutic agents intramuscularly in response to processor-generated trigger signals combined with safety interlock confirmations, providing autonomous emergency treatment without requiring manual user intervention during anaphylactic episodes.

In one aspect, variations of the disclosed wearable autonomous anaphylaxis treatment system may include layered architectural configurations with integrated sensing, processing, actuation, and power management components. The system comprises a wearable therapeutic unit configured for body placement and incorporating distinct functional layers including skin-facing adhesive interfaces with integrated contact sensors, fluidic layers containing drug reservoirs and mechanical actuators, electronics layers housing processors and sensor interfaces, and outer layers providing power sources and wireless communication modules. Physiological sensors distributed across the system architecture communicate with central processing components to enable concurrent monitoring of multiple physiological parameters. The processor detects concurrent physiological changes across multiple parameters indicative of anaphylaxis, generates trigger signals upon detection, and activates mechanical actuators to deliver therapeutic agents when safety conditions are confirmed.

In one aspect, variations of the disclosed method of autonomously treating anaphylaxis may include continuous physiological monitoring, baseline calibration procedures, multi-system condition detection, confidence scoring analysis, and automated therapeutic intervention. The method involves monitoring respiratory function, cardiovascular function, and autonomic function through multiple physiological sensors to generate continuous sensor data. Baseline physiological states are established through analysis of sensor data over calibration periods, enabling detection of deviations through real-time comparisons. Respiratory distress conditions are identified when sensor data indicates breathing rate increases, irregular patterns, or characteristic respiratory sounds. Cardiovascular shock conditions are identified when sensor data indicates heart rate increases, heart rate variability decreases, or blood perfusion reductions. Confidence scores are generated based on concurrent presence of multiple identified conditions, with injection trigger signals generated when confidence scores exceed predetermined thresholds. Therapeutic agents are delivered intramuscularly through wearable injection mechanisms in response to trigger signals combined with skin contact verification, providing autonomous emergency treatment during anaphylactic episodes.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a cross-sectional view of a spring-actuated injection mechanism of a wearable autonomous anaphylaxis treatment device according to some embodiments;

FIG. 2 illustrates a flowchart diagram of overall system operation process for the wearable autonomous anaphylaxis treatment device of FIG. 1, according to some embodiments;

FIG. 3 illustrates a flowchart diagram of the multi-system anaphylaxis detection algorithm process for the wearable autonomous anaphylaxis treatment device of FIG. 1, according to some embodiments;

FIG. 4 illustrates a flowchart diagram of the safety interlock and alert sequence process for the wearable autonomous anaphylaxis treatment device of FIG. 1, according to some embodiments; and

FIG. 5 illustrates a block diagram of the layered system architecture of the wearable autonomous anaphylaxis treatment device of FIG. 1, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

For clarity and consistency, the same reference numerals will be used throughout the detailed description to refer to the same or corresponding components across the various figures. When a particular component is discussed while referring to a figure different from the one in which the component first appears, the reference numeral and original figure will be cited for clarity.

Terms such as “inner,” “outer,” “first,” “second,” “proximal,” and “distal” may be used in the following description for clarity with respect to the orientation of components as shown in the figures. These terms are not intended to be limiting and may be interpreted relative to the position or orientation of the wearable autonomous anaphylaxis treatment device in actual use, which may vary.

Unless otherwise specified, the singular forms “a,” “an,” and “the” include plural referents. Components may be described functionally rather than structurally where appropriate for clarity. Any features or configurations disclosed as being optional or alternative may be implemented individually or in any suitable combination, as would be understood by one of ordinary skill in the art.

The disclosed wearable autonomous anaphylaxis treatment device may include housing materials formed from biocompatible polymeric, elastomeric, or composite materials that provide structural integrity while maintaining skin compatibility and flexibility requirements compared to conventional rigid medical devices. The systems may be configured to eliminate manual intervention procedures through integrated autonomous detection and actuation while maintaining controlled therapeutic delivery functionality during normal operational cycles. In some embodiments, alternative material formulations such as medical-grade silicones, thermoplastic polyurethanes, or biocompatible polymer composites may be used to achieve similar strength characteristics and skin compatibility properties.

The wearable autonomous anaphylaxis treatment device may comprise physiological sensor elements and multi-system detection components that enable accurate anaphylaxis identification without requiring manual symptom recognition procedures. The detection mechanisms may be configured to provide reliable pattern identification through baseline calibration, multi-parameter analysis, and confidence scoring application, eliminating the time-consuming and error-prone characteristics typically associated with traditional manual symptom assessment systems. The sensor elements may be positioned within sections of the device structure and may feature continuous monitoring characteristics to provide physiological data collection during surveillance cycles. The detection components may be configured to engage exclusively with sensor data streams, creating diagnostic determinations that resist false positive conditions under normal physiological variations while permitting accurate detection without manual interpretation.

The wearable autonomous anaphylaxis treatment device may also include integrated safety interlock mechanisms positioned within the system architecture, dimensioned to provide contact verification and user alert capabilities for controlled intervention, conscious user notification, or emergency override requirements. These integrated components may provide reliable safety surfaces while eliminating the complexity and delay challenges associated with separate monitoring devices and manual actuation systems. The safety components may be optimized to accommodate various user requirements while maintaining the structural integrity and operational characteristics of the overall assembly.

The wearable autonomous anaphylaxis treatment device may be configured to enable multi-stage safety verification during operation, maintaining proper therapeutic delivery control without requiring specialized medical training or extensive user intervention procedures. The systems may be configured to engage through automated detection and safety confirmation, providing adaptability in emergency applications and operational configurations. The multi-stage safety connections may be configured to resist inappropriate deployment under various physiological conditions while permitting consistent operational characteristics and reliable intervention without degradation of the safety features.

The wearable autonomous anaphylaxis treatment device may include multiple sensor configurations designed for different monitoring approaches, including acoustic sensors for respiratory assessment and optical sensors for cardiovascular evaluation situations. Each system type may incorporate the same detection principles while providing specialized sensing configurations to address specific physiological parameter requirements.

In use, individuals with anaphylaxis risk or caregivers may position the wearable autonomous anaphylaxis treatment device according to body placement requirements and rely on the autonomous mechanisms to create controlled intervention functionality without extensive medical training considerations. The systems may operate by monitoring physiological parameters according to baseline requirements and activating the injection mechanism when anaphylaxis conditions are detected. The sensor elements engage with the detection components through continuous processing, creating diagnostic confidence that maintains proper emergency response during critical reaction cycles. The injection mechanism operates through automated activation rather than manual operation, eliminating procedural complexity and enabling consistent performance throughout the emergency intervention cycle.

An individual with severe food allergies may utilize the wearable autonomous anaphylaxis treatment device to address life-threatening reaction risks commonly experienced when accidental allergen exposure occurs during daily activities. The elimination of manual recognition and injection procedures prevents the treatment delays and panic responses that often result from traditional auto-injector techniques during severe allergic reactions. The consistent detection characteristics function across multiple symptom presentations, improving survival outcomes during anaphylactic episodes and reducing mortality risks associated with delayed or absent epinephrine administration.

A parent of a child with severe allergies working in school environments and recreational activities may benefit from the system's ability to maintain continuous monitoring during various situations such as meal times, outdoor activities, and social events. The autonomous detection technology maintains protective surveillance without requiring the constant vigilance that could restrict childhood experiences, while the automated intervention eliminates the response limitations parents often face when managing unpredictable allergic emergencies in children unable to self-administer treatment.

A healthcare facility treating high-risk patients may employ the wearable autonomous anaphylaxis treatment device when monitoring individuals undergoing immunotherapy or medication trials that carry anaphylaxis risks. The consistent operational characteristics reduce monitoring complexity during treatment protocols, while the reliable intervention ensures emergency response occurs immediately during adverse reactions. The elimination of manual recognition requirements prevents healthcare personnel from experiencing response variations during simultaneous patient care scenarios, improving treatment safety consistency with clinical protocols.

It should be noted that the use cases described above are merely illustrative examples of how the wearable autonomous anaphylaxis treatment device may be utilized, and the practical applications are not limited to these specific scenarios. The autonomous detection design and integrated intervention features make the system suitable for a wide variety of medical applications where immediate response, continuous monitoring, or automated emergency treatment requirements provide advantages over conventional manual auto-injector systems.

The disclosed wearable autonomous anaphylaxis treatment device provides a system that offers an autonomous alternative to conventional manual approaches configured to address the critical challenges of time-sensitive anaphylaxis intervention applications. By incorporating multi-modal physiological sensing technology, integrated artificial intelligence detection components, and automated injection systems, the device enables controlled emergency intervention functionality without manual recognition dependencies while eliminating the response delays associated with traditional manually-activated auto-injector techniques. Unlike conventional epinephrine delivery that relies on symptom recognition and creates treatment variations across emergency response capabilities, this configuration provides consistent intervention through autonomous processes and incorporates intelligent design elements to improve survival outcomes, enabling anaphylaxis treatment to proceed effectively in a variety of daily life, pediatric, healthcare, and emergency applications.

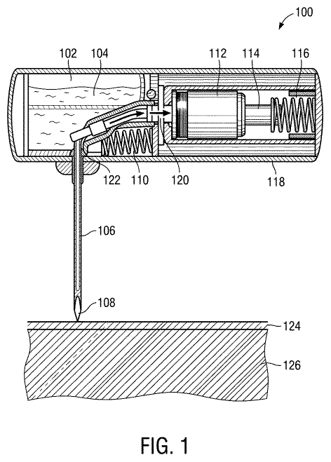

FIG. 1 illustrates a cross-sectional view of an injection mechanism 100 of a wearable autonomous anaphylaxis treatment device according to one embodiment of the disclosed invention. The injection mechanism 100 represents a dual-compartment drug delivery assembly that integrates therapeutic agent storage, mechanical actuation components, needle deployment mechanisms, and intramuscular (IM) delivery functionality within a unified structure configured for wearable implementation.

The injection mechanism 100 comprises a housing 118 configured as a cylindrical structure containing dual compartments that separate the plunger 112 from the drug reservoir 102. The housing 118 connects to the various internal components through mounting interfaces that maintain proper component alignment and spatial separation between compartments. The dual-compartment configuration of the housing 118 enables independent positioning of the plunger 112 in a first compartment and the drug reservoir 102 in a second compartment, providing mechanical isolation that facilitates the needle reorientation mechanism described below. The cylindrical geometry of the housing 118 provides the structural framework that supports both the actuation pathway from the spring 110 through the plunger 112 and the separate drug storage and delivery pathway through the drug reservoir 102 and needle 106. The enclosed configuration of the housing 118 protects the internal components from contamination and mechanical damage during wearable operation. The housing 118 functions by maintaining the spatial relationships between components while providing the reaction surfaces required for spring 110 compression and expansion and enabling the needle deployment sequence, allowing the injection mechanism 100 to operate as an integrated assembly.

The drug reservoir 102 positioned within the second compartment of the housing 118 comprises a sealed chamber configured to contain the therapeutic agent 104 under controlled storage conditions. The drug reservoir 102 connects to the housing 118 through sealed interfaces that maintain the sterility and stability of the therapeutic agent 104 during extended wear periods while enabling fluid communication with the needle 106 through the valve 122. The configuration of the drug reservoir 102 provides volumetric capacity sufficient to store a single therapeutic dose while maintaining the compact form factor required for wearable applications. The sealed chamber construction prevents contamination and degradation of the therapeutic agent 104 through environmental exposure, enabling the injection mechanism 100 to maintain therapeutic efficacy throughout the operational lifespan of the device. The drug reservoir 102 functions by maintaining the therapeutic agent 104 in a ready state within the second compartment until mechanical actuation initiates delivery, eliminating the preparation delays associated with conventional multi-component drug delivery systems while remaining spatially separated from the plunger actuation compartment.

The therapeutic agent 104 contained within the drug reservoir 102 comprises the active pharmaceutical compound configured for emergency anaphylaxis treatment. The therapeutic agent 104 fills the internal volume of the drug reservoir 102, creating a fluid pathway that enables rapid expulsion during actuation. In some embodiments, the therapeutic agent 104 comprises epinephrine in concentrations ranging from 0.15 milligrams to 0.3 milligrams depending on patient weight and treatment protocols. The liquid formulation of the therapeutic agent 104 enables immediate availability upon actuation without requiring reconstitution procedures, providing the rapid intervention timeframes essential for anaphylaxis emergencies. The therapeutic agent 104 remains in direct fluid communication with the needle 106 through the valve 122, creating a complete fluid pathway from storage to delivery that functions during the injection sequence.

The valve 122 positioned between the drug reservoir 102 and the needle 106 comprises a rupturable seal configured to maintain separation between the therapeutic agent 104 and the needle 106 during storage while enabling fluid communication during actuation. The valve 122 connects to both the drug reservoir 102 and the needle 106 through sealed interfaces that prevent leakage during normal wear conditions and during the needle reorientation sequence. The rupturable configuration of the valve 122 enables the seal to fail under controlled pressure conditions generated during actuation, creating the fluid pathway along the injection path 120 from the drug reservoir 102 through the needle 106. This controlled rupture mechanism eliminates the complexity of mechanical valve systems while ensuring reliable fluid communication establishment during emergency intervention regardless of needle position during deployment. The valve 122 functions by maintaining storage integrity through membrane strength while providing predictable rupture characteristics under actuation forces, enabling consistent therapeutic delivery without valve malfunction risks during needle reorientation and deployment.

The needle 106 connected to the valve 122 comprises a hollow tubular structure configured to reorient from a non-penetrating configuration to a penetrating configuration and deliver the therapeutic agent 104 into the muscle tissue 126. In the resting state prior to actuation, the needle 106 is retained in a non-penetrating configuration relative to the skin surface 124. The non-penetrating configuration comprises the needle 106 positioned in at least one of a folded orientation, angled orientation, or L-shaped orientation where the needle axis is not aligned for direct skin penetration, preventing inadvertent tissue contact during device wear and handling. Upon actuation release, the needle 106 reorients from the non-penetrating configuration and translates into a penetrating position where the needle 106 extends toward and through the skin surface 124 to reach the muscle tissue 126 below. The needle 106 connects to the valve 122 at a proximal end and terminates at the needle tip 108 at a distal end. The hollow bore of the needle 106 creates the fluid conduit that receives the therapeutic agent 104 from the drug reservoir 102 and directs the fluid through the needle tip 108 into the tissue. The length of the needle 106 provides sufficient penetration depth to reach the muscle tissue 126 below the skin surface 124 when deployed to the penetrating position, enabling intramuscular delivery rather than subcutaneous administration. In some embodiments, the needle 106 comprises lengths ranging from 1.17 centimeters to 2.5 centimeters to accommodate varying tissue depths across different body placement locations. The needle 106 functions by reorienting from a safe non-penetrating storage configuration to an active penetrating configuration during the actuation sequence, creating a penetration pathway through the skin surface 124 into the muscle tissue 126 while providing a fluid conduit for therapeutic agent 104 delivery, enabling the rapid absorption rates associated with intramuscular administration for emergency anaphylaxis treatment.

The needle tip 108 positioned at the distal end of the needle 106 comprises a sharpened point configured to penetrate the skin surface 124 and muscle tissue 126 with controlled force application when the needle 106 has reoriented to the penetrating position. The needle tip 108 forms the terminal structure of the needle 106, creating the initial tissue penetration during the injection sequence following needle deployment. The sharpened geometry of the needle tip 108 reduces the force requirements for tissue penetration, enabling the deployment mechanism to drive the needle 106 through the skin surface 124 and into the muscle tissue 126 without excessive impact forces that could cause tissue damage or user discomfort. The needle tip 108 functions by concentrating the deployment forces into a localized penetration point after needle reorientation, creating a tissue pathway for the needle 106 while minimizing the tissue disruption associated with larger diameter penetrations.

The plunger 112 positioned within the first compartment of the housing 118 separate from the drug reservoir 102 comprises a movable element configured to initiate the injection sequence through stored mechanical energy release. The plunger 112 connects to the plunger rod 114 through a mechanical interface that transfers forces from the spring 110 to the plunger 112. The spatial separation of the plunger 112 within the first compartment from the drug reservoir 102 within the second compartment enables the dual-compartment architecture where plunger actuation triggers both needle deployment and therapeutic agent expulsion through coordinated mechanical action. The movable configuration of the plunger 112 enables axial displacement within the first compartment in response to forces applied by the spring 110 through the plunger rod 114. The plunger 112 functions by converting the mechanical energy stored in the spring 110 into deployment energy that initiates the needle reorientation sequence and drives therapeutic agent 104 expulsion from the drug reservoir 102 through mechanical coupling, generating the coordinated actions required for successful intramuscular injection following autonomous detection.

The plunger rod 114 extending from the plunger 112 toward the rear spring 116 comprises a connecting element configured to transfer actuation forces from the spring 110 to the plunger 112 and through mechanical linkages to the needle deployment mechanism. The plunger rod 114 connects to the plunger 112 at a first end and engages with the spring 110 at a second end. The construction of the plunger rod 114 resists bending or buckling under the forces generated by the spring 110 during actuation, maintaining force alignment throughout the injection sequence. The plunger rod 114 may incorporate or connect to mechanical linkages that couple plunger motion to needle reorientation, enabling the stored energy release to accomplish both needle deployment from non-penetrating to penetrating configuration and therapeutic agent expulsion. The plunger rod 114 functions by maintaining mechanical connection between the spring 110 and the plunger 112 while enabling force transmission to accomplish coordinated needle deployment and drug delivery, ensuring the mechanical energy stored in the spring 110 converts into both needle reorientation and therapeutic agent 104 delivery.

The spring 110 positioned within the first compartment adjacent to the plunger 112 comprises a coiled elastic element configured to store mechanical energy and drive the plunger 112 during injection actuation. The spring 110 connects to the plunger rod 114 at a first end and engages with the housing 118 at a second end. The coiled configuration of the spring 110 enables compression during loading operations and expansion during actuation, converting stored elastic potential energy into kinetic energy that drives the plunger 112. The spring constant of the spring 110 generates sufficient force to accomplish needle reorientation from the non-penetrating configuration to the penetrating position, rupture the valve 122, expel the therapeutic agent 104 from the drug reservoir 102, and drive the needle 106 through the skin surface 124 into the muscle tissue 126 once deployed. In some embodiments, the spring 110 generates forces approaching 35 Newtons to ensure reliable needle deployment and intramuscular penetration across varying tissue densities and body placement locations. The spring 110 functions by maintaining stored energy in a compressed state until actuation release, then rapidly expanding to drive the plunger 112 through the complete injection sequence including needle reorientation and therapeutic delivery within timeframes suitable for emergency intervention.

The rear spring 116 positioned at the opposite end of the housing 118 from the drug reservoir 102 comprises an additional elastic element configured to provide mechanical functionality during the injection sequence. The rear spring 116 connects to the housing 118 and engages with internal components to provide controlled mechanical response during actuation. The coiled configuration of the rear spring 116 enables compression and expansion cycles that support the overall injection mechanism 100 operation. In some embodiments, the rear spring 116 assists with needle deployment through mechanical linkage to the needle reorientation mechanism. In some embodiments, the rear spring 116 provides needle retraction functionality following therapeutic agent 104 delivery, enabling the needle 106 to withdraw from tissue contact after injection completion. The rear spring 116 functions by providing mechanical assistance during specific phases of the injection sequence including needle deployment or retraction, contributing to the controlled delivery characteristics of the injection mechanism 100.

The injection path 120 indicated by the arrow extending from the drug reservoir 102 through the needle 106 represents the fluid flow direction during therapeutic agent 104 delivery. The injection path 120 follows the hollow bore of the needle 106 from the valve 122 to the needle tip 108, creating the route through which the therapeutic agent 104 travels during expulsion following needle deployment to the penetrating position. The configuration of the injection path 120 accommodates the needle reorientation from non-penetrating to penetrating position, maintaining fluid communication throughout the deployment sequence. The injection path 120 functions by directing the therapeutic agent 104 from the storage volume of the drug reservoir 102 to the delivery location within the muscle tissue 126 after needle reorientation, completing the drug delivery sequence initiated by spring 110 actuation.

The skin surface 124 shown at the bottom of FIG. 1 represents the external tissue layer through which the needle 106 penetrates during injection actuation following reorientation to the penetrating position. The skin surface 124 forms the boundary between the external environment and the internal tissue structures, creating the barrier encountered by the needle tip 108 during penetration after needle deployment. The skin surface 124 connects to the underlying muscle tissue 126 through intermediate tissue layers not separately depicted in FIG. 1. The mechanical properties of the skin surface 124 including thickness, elasticity, and tensile strength influence the penetration forces required for needle 106 insertion following reorientation. The skin surface 124 functions by providing the anatomical interface where the injection mechanism 100 delivers the therapeutic agent 104 during emergency intervention after the needle 106 has deployed from the non-penetrating configuration.

The muscle tissue 126 positioned below the skin surface 124 represents the target tissue for therapeutic agent 104 delivery during intramuscular injection. The muscle tissue 126 comprises the vascularized tissue structure that enables rapid therapeutic agent 104 absorption into systemic circulation. The depth of the muscle tissue 126 below the skin surface 124 determines the required needle 106 length for successful intramuscular delivery following needle deployment. The vascular properties of the muscle tissue 126 provide the absorption characteristics required for rapid epinephrine action during anaphylaxis emergencies. The muscle tissue 126 functions by receiving the therapeutic agent 104 delivered through the needle 106 after needle reorientation and facilitating rapid absorption into the bloodstream, enabling the systemic therapeutic effects required for anaphylaxis treatment within the critical intervention timeframe.

In some embodiments, the dual-compartment structure of the housing 118 comprises separate sealed chambers with the plunger 112 disposed in a first compartment and the drug reservoir 102 disposed in a second compartment, with mechanical coupling between compartments enabling plunger actuation to trigger needle deployment. In some embodiments, the needle 106 in the non-penetrating configuration comprises a folded orientation where the needle folds against the housing 118 parallel to the skin surface 124. In some embodiments, the needle 106 in the non-penetrating configuration comprises an angled orientation where the needle axis forms an angle relative to the intended penetration direction. In some embodiments, the needle 106 in the non-penetrating configuration comprises an L-shaped orientation where the needle extends perpendicular to the penetration axis. In some embodiments, the needle reorientation mechanism comprises mechanical linkages connecting the plunger rod 114 to the needle 106, where plunger 112 displacement causes the linkages to rotate or translate the needle 106 from the non-penetrating configuration to the penetrating position. In some embodiments, the needle deployment is assisted by gravitational force when the device is oriented with the needle positioned above the actuation mechanism. In some embodiments, the needle deployment is assisted by spring force from dedicated deployment springs separate from the main spring 110. In some embodiments, the needle deployment comprises combined assistance from gravity, spring force, and mechanical linkage acting cooperatively to achieve reliable needle reorientation. In some embodiments, the needle deployment mechanism is subject to contact sensor verification as described in subsequent figures, preventing needle deployment unless skin contact is confirmed. In some embodiments, the needle deployment mechanism is subject to lockout mechanisms as described in subsequent figures, preventing repeated deployment after initial therapeutic delivery.

In some embodiments, the drug reservoir 102 comprises materials selected from medical-grade polymers, glass, or composite materials configured to maintain therapeutic agent 104 stability during extended storage periods ranging from six months to two years. In some embodiments, the therapeutic agent 104 comprises epinephrine concentrations of 0.15 milligrams for pediatric applications or 0.3 milligrams for adult applications, with formulations including stabilizers and preservatives to maintain potency.

In some embodiments, the needle 106 comprises stainless steel, titanium, or polymer materials with hollow bore diameters ranging from 0.3 millimeters to 0.8 millimeters to accommodate varying flow rate requirements. In some embodiments, the needle 106 features beveled needle tips 108, tri-bevel configurations, or specialized geometries to reduce penetration forces and tissue trauma.

In some embodiments, the spring 110 comprises stainless steel coil springs, compression springs, or wave springs configured to generate forces ranging from 25 Newtons to 45 Newtons for needle deployment and intramuscular penetration. In some embodiments, the plunger 112 incorporates coupling mechanisms including cams, levers, or linkages that convert linear plunger displacement into rotational or translational needle movement for reorientation.

In some embodiments, the housing 118 comprises injection-molded polymers, machined metals, or composite materials configured to withstand internal forces generated during actuation while maintaining lightweight characteristics for wearable applications and providing compartment separation. In some embodiments, the valve 122 comprises burst discs, frangible membranes, or mechanical valve assemblies configured to open at predetermined pressure thresholds ranging from 50 psi to 200 psi while maintaining seal integrity during needle reorientation.

In some embodiments, the injection mechanism 100 incorporates safety mechanisms including needle shields covering the needle 106 in the non-penetrating configuration, trigger locks preventing inadvertent plunger release, or activation interlocks requiring contact sensor confirmation before enabling deployment. In some embodiments, the rear spring 116 provides needle retraction functionality, enabling the needle 106 to withdraw into the housing 118 following therapeutic agent 104 delivery by reorienting from the penetrating position back toward the non-penetrating configuration to prevent continued tissue contact.

In some embodiments, the injection mechanism 100 comprises a microneedle array alternative to the reorienting needle 106. The microneedle array comprises a plurality of needles arranged in an array configuration with individual needle lengths sufficient to achieve intramuscular penetration depths. The mechanical actuator for the microneedle array comprises thermally expandable material positioned adjacent to the drug reservoir 102, wherein thermal activation causes the thermally expandable material to expand and pressurize the drug reservoir 102, driving the therapeutic agent 104 through the microneedle array into the muscle tissue 126. In some embodiments, the thermally expandable material comprises phase-change materials, thermally responsive polymers, or shape memory alloys activated through resistive heating elements. In some embodiments, the microneedle array comprises needle lengths ranging from 1.5 millimeters to 3 millimeters to achieve intramuscular delivery depending on body placement location and tissue depth.

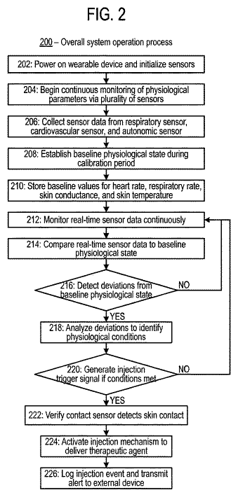

FIG. 2 illustrates a flowchart diagram of the overall system operational sequence 200 for the wearable autonomous anaphylaxis treatment device showing the temporal sequence from initialization through therapeutic intervention according to the embodiment described in FIG. 1. The operational sequence 200 represents the algorithmic flow executed by the processor to enable continuous physiological monitoring, baseline establishment, deviation detection, and autonomous injection actuation without requiring manual user intervention.

The sequence 200 begins at operation 202, which involves powering on the wearable device and initializing the plurality of physiological sensors. Operation 202 comprises the startup routine where the processor activates electrical connections to the respiratory sensor, cardiovascular sensor, and autonomic sensor while performing self-diagnostic checks to verify sensor functionality and communication pathway integrity. The processor at operation 202 may be implemented as a microcontroller unit with integrated peripherals, a system-on-chip combining central processing unit with dedicated sensor interface hardware, or an application-specific integrated circuit optimized for low-power biomedical signal processing. The processor may comprise digital signal processing capabilities for real-time filtering and feature extraction, hardware multiply-accumulate units for accelerating correlation calculations, and dedicated analog-to-digital converter channels for simultaneous multi-sensor sampling. The processor executes initialization routines stored in non-volatile memory including flash memory or one-time programmable read-only memory, configuring analog-to-digital converter sampling rates, establishing baseline clock frequencies selected to balance processing performance against power consumption, allocating memory buffers in random access memory for circular buffer data storage, and verifying wireless communication module connectivity through handshake protocols. The memory architecture supporting operation 202 may include multi-level memory hierarchies with fast static random access memory for processor registers and cache, dynamic random access memory for active data buffers, and non-volatile flash memory for firmware storage and persistent baseline data. Operation 202 functions by transitioning the device from an inactive storage state to an operational monitoring state, ensuring all sensor elements respond within specified voltage ranges and communication pathways between sensors and processor operate within timing specifications before proceeding to continuous monitoring activities. The initialization sequence prevents false triggering from incomplete sensor stabilization periods and establishes known operational states for subsequent processing routines through register initialization and peripheral configuration.

Following initialization, the sequence advances to operation 204, which involves beginning continuous monitoring of physiological parameters via the plurality of sensors. Operation 204 comprises activating persistent sensor data streams where the respiratory sensor begins detecting breathing rate, breathing pattern, and acoustic respiratory features through acoustic transducer signals, the cardiovascular sensor begins measuring heart rate, heart rate variability, and blood perfusion parameters through photoplethysmography or electrocardiogram signal processing, and the autonomic sensor begins monitoring skin conductance through galvanic skin response measurement and skin temperature changes through thermistor resistance values. The continuous monitoring at operation 204 operates without interruption throughout device wear periods, creating unbroken data streams that enable real-time physiological assessment regardless of user activity or environmental conditions. The processor during operation 204 maintains active data acquisition channels configured through peripheral registers, implementing interrupt-driven sampling where analog-to-digital converter completion triggers interrupt service routines that transfer digitized samples to memory buffers without processor intervention. In some embodiments, the processor implements circular buffer architectures where recent sensor data overwrites oldest data, maintaining rolling windows of physiological history spanning time periods from 10 seconds to 10 minutes depending on computational routine requirements for pattern detection and temporal correlation analysis. Operation 204 functions by establishing persistent surveillance that detects anaphylaxis onset regardless of when allergen exposure occurs, eliminating monitoring gaps associated with periodic assessment approaches and providing protection during sleep periods, physical activities, or situations where manual symptom recognition proves unreliable due to cognitive impairment during severe reactions.

The sequence proceeds to operation 206, which involves collecting sensor data from the respiratory sensor, cardiovascular sensor, and autonomic sensor. Operation 206 comprises the processor receiving digitized sensor signals through sensor interfaces that will be discussed in subsequent figures, accumulating physiological measurements over time intervals suitable for pattern analysis and statistical characterization. The data collection at operation 206 captures breathing rate values measured in breaths per minute calculated through respiratory cycle detection logic that identifies inspiration and expiration phases, acoustic features extracted from respiratory sounds through fast Fourier transform analysis identifying frequency content and temporal patterns characteristic of wheeze or stridor, heart rate measurements in beats per minute calculated through peak detection routines applied to photoplethysmography or electrocardiogram waveforms, heart rate variability metrics calculated through inter-beat interval analysis quantifying autonomic nervous system balance, blood perfusion proxies derived from photoplethysmography signal amplitudes indicating peripheral circulation status, skin conductance readings in microsiemens calculated through voltage-to-conductance conversion indicating autonomic nervous system activity and sympathetic arousal, and temperature measurements in degrees Celsius calculated through thermistor resistance-to-temperature conversion tracking peripheral circulation changes and vasomotor responses. The processor aggregates these multi-parameter measurements into time-stamped data structures stored in memory, creating datasets spanning sufficient durations to enable baseline calculation through statistical analysis and deviation analysis through temporal comparison. In some embodiments, the data structures comprise arrays of sensor values with associated timestamps enabling chronological reconstruction of physiological changes. In some embodiments, the data structures comprise linked lists enabling efficient insertion and deletion during rolling window updates where oldest measurements are discarded as new measurements arrive. In some embodiments, the data structures comprise ring buffers enabling constant-time access to recent measurements without memory reallocation overhead. Operation 206 functions by accumulating comprehensive physiological data into unified datasets that enable the processor to assess concurrent changes across multiple physiological parameters rather than evaluating isolated measurements, providing the information foundation required for multi-system anaphylaxis detection through correlation analysis and pattern matching routines.

Following data collection, the sequence advances to operation 208, which involves establishing the baseline physiological state during a calibration period. Operation 208 comprises the processor analyzing accumulated sensor data from operation 206 through statistical calculation routines that compute representative values characterizing the user's normal physiological parameters in the absence of anaphylaxis. The processor executes calibration routines implemented in firmware that may include calculating mean values through summation and division operations, calculating median values through sorting and selection procedures, or calculating moving averages through recursive filters that weight recent measurements more heavily than historical measurements. In some embodiments, the processor calculates variance or standard deviation metrics through sum-of-squares calculations that quantify normal physiological variability, enabling subsequent deviation detection to distinguish statistically changes from normal fluctuations. In some embodiments, the processor implements outlier detection logic including median absolute deviation calculations or z-score filtering that identify and exclude measurements exceeding statistical thresholds, preventing transient disturbances or sensor artifacts from biasing baseline calculations. In some embodiments, the baseline establishment at operation 208 occurs over calibration periods ranging from 5 minutes to 3 hours depending on computational routine requirements for statistical confidence and the magnitude of individual variability patterns. The processor during operation 208 generates personalized baseline values for heart rate reflecting individual resting heart rate and typical daily variations. In some embodiments, baseline heart rate values span 40 beats per minute to 100 beats per minute across user populations. In some embodiments, baseline respiratory rate accounts for normal breathing patterns during rest and activity spanning 8 breaths per minute to 20 breaths per minute. In some embodiments, baseline temperature indicates normal peripheral circulation under ambient conditions spanning 28 degrees Celsius to 34 degrees Celsius. Operation 208 functions by creating user-specific reference values against which subsequent measurements are compared, accommodating individual variations in physiological parameters that differ substantially between users based on age, fitness level, medication use including beta-blockers affecting heart rate or anticholinergics affecting sweating, and baseline health status including cardiovascular fitness or thyroid function rather than relying on population-averaged reference ranges that may generate false positives or false negatives for individuals at distribution extremes.

The sequence continues to operation 210, which involves storing the baseline values for heart rate, respiratory rate, skin conductance, and skin temperature in memory accessible to the processor. Operation 210 comprises the processor writing calculated baseline values to non-volatile memory where they remain accessible throughout subsequent monitoring cycles and persist through power cycling events or device removal and reapplication. In some embodiments, the memory utilized at operation 210 includes flash memory organized in pages or blocks requiring erase-before-write operations. In some embodiments, the memory comprises electrically erasable programmable read-only memory enabling byte-level updates. In some embodiments, the memory comprises battery-backed static random access memory maintaining data integrity during power interruptions through backup power sources. In some embodiments, the non-volatile memory implements wear-leveling procedures that distribute write operations across memory cells to prevent premature failure of frequently-updated locations. The stored baseline dataset at operation 210 may include not only mean baseline values but also associated statistical parameters including standard deviations quantifying expected variability, confidence intervals defining ranges containing true population parameters with specified probabilities, or percentile ranges indicating thresholds for deviations. In some embodiments, the processor stores metadata associated with baseline values including timestamps indicating calculation time, validity flags indicating whether sufficient data supported calculation, or version identifiers enabling processing routine updates to recognize baseline data formats. Operation 210 functions by creating persistent reference datasets that enable deviation detection without requiring recalibration after each device activation, improving operational continuity during multi-day wear scenarios and eliminating recalibration delays when users remove and reapply the device for hygiene or comfort adjustments.

Following baseline storage, the sequence enters operation 212, which involves monitoring real-time sensor data continuously. Operation 212 comprises the processor receiving ongoing sensor data streams from the physiological sensors while the user conducts daily activities including eating, exercising, sleeping, or engaging in social or occupational activities where allergen exposure may occur. The continuous monitoring at operation 212 operates as a persistent background procedure where the processor maintains active sensor connections and processes incoming data without requiring user interaction or manual activation. The real-time monitoring at operation 212 captures physiological dynamics as they evolve, enabling the processor to detect anaphylaxis onset within timeframes supporting early intervention. Operation 212 functions by maintaining constant physiological surveillance that provides protection throughout wear periods, capturing anaphylaxis development during contexts where symptom recognition proves difficult including sleep, physical activity inducing physiological changes that mask early symptoms, or high-stress situations where attention focuses elsewhere.

The sequence advances to operation 214, which involves comparing the real-time sensor data to the baseline physiological state established at operation 208. Operation 214 comprises the processor calculating deviation metrics by subtracting stored baseline values from current sensor measurements, generating quantitative assessments of physiological changes relative to individual norms. The comparison at operation 214 produces deviation values for each monitored parameter including heart rate deviation calculated as current heart rate minus baseline heart rate, respiratory rate deviation representing breathing rate change from baseline, skin conductance deviation indicating autonomic response relative to baseline, and temperature deviation tracking peripheral circulation changes. In some embodiments, the processor calculates absolute deviations in original measurement units preserving direct physiological interpretation. In some embodiments, the processor calculates percentage deviations relative to baseline values enabling standardized comparison across parameters with different measurement scales. In some embodiments, the processor calculates normalized deviations expressed in standard deviation units relative to baseline variability captured during calibration, enabling statistical assessment. Operation 214 functions by converting raw sensor measurements into deviation metrics that indicate physiological changes, enabling the processor to identify departures from normal conditions through quantitative thresholds rather than subjective symptom assessment, and providing the deviation data required for subsequent anaphylaxis pattern recognition.

The sequence then reaches decision point 216, which determines whether deviations from the baseline physiological state are detected. Decision point 216 comprises the processor evaluating whether deviation metrics calculated at operation 214 exceed predetermined threshold values indicating physiological changes warranting further anaphylaxis analysis. In some embodiments, the threshold evaluation at decision point 216 compares absolute deviation magnitudes against fixed thresholds expressed in original measurement units. In some embodiments, the threshold evaluation assesses percentage deviations against proportional thresholds accounting for relative changes. In some embodiments, the threshold evaluation evaluates normalized deviations against statistical thresholds such as values exceeding two or three standard deviations from baseline. When decision point 216 determines that deviations remain below threshold values indicating normal physiological variation, the sequence returns to operation 212 via the feedback pathway to continue monitoring real-time sensor data, creating a continuous surveillance loop. When decision point 216 determines that one or more deviation metrics exceed threshold values, the sequence advances to operation 218 for detailed anaphylaxis evaluation. Decision point 216 functions by filtering routine physiological variations from potentially changes, preventing unnecessary computational processing during normal activities while ensuring abnormal patterns receive immediate analysis, and balancing sensitivity for early anaphylaxis detection against specificity to avoid false positive interventions.

Following detection of deviations, the sequence advances to operation 218, which involves analyzing the deviations to identify physiological conditions. Operation 218 comprises the processor executing detection logic that evaluates whether detected deviations match patterns consistent with respiratory distress condition, cardiovascular shock condition, or autonomic activation condition as detailed in the multi-system detection method shown in a subsequent figure. The analysis at operation 218 processes deviation data through pattern classification routines, multi-parameter correlation assessments, and temporal trend evaluations to determine condition presence and severity. In some embodiments, the processor during operation 218 applies rules-based decision logic implementing explicit conditional statements that evaluate sensor combinations against predetermined criteria. In some embodiments, the processor applies machine learning classifiers trained on labeled physiological data distinguishing anaphylaxis from non-anaphylaxis states. In some embodiments, the processor applies hybrid decision systems combining rules-based thresholds for high-confidence cases with probabilistic classification for ambiguous presentations. Operation 218 functions by transforming deviation metrics into condition identifications that enable the processor to recognize multi-system anaphylaxis patterns rather than isolated parameter changes, distinguishing true anaphylactic responses characterized by concurrent respiratory and cardiovascular involvement from anxiety reactions producing heart rate elevation without respiratory compromise, exercise-induced changes featuring coordinated cardiovascular and respiratory responses with different temporal characteristics, or environmental temperature changes affecting skin parameters without cardiovascular or respiratory involvement.

The sequence then reaches decision point 220, which determines whether to generate the injection trigger signal based on whether anaphylaxis conditions are met. Decision point 220 comprises the processor evaluating whether the identified conditions from operation 218 satisfy criteria for autonomous intervention including confidence score exceeding predetermined threshold and presence of at least two physiological conditions from respiratory distress, cardiovascular shock, or autonomic activation. When decision point 220 determines conditions are not met, indicating either absence of anaphylaxis or insufficient confidence in detection, the sequence returns to operation 212 via the feedback pathway to continue monitoring without intervention. When decision point 220 determines conditions are satisfied, indicating high confidence anaphylaxis detection across multiple physiological systems, the sequence advances to operation 222 to initiate intervention sequence. Decision point 220 functions by implementing the multi-system requirement that reduces false positive rates compared to single-parameter triggers, applying confidence thresholds that balance early intervention benefits against intervention risks for uncertain detections, and enabling autonomous therapeutic decisions without requiring manual user confirmation during emergencies where decision-making capacity may be impaired.

Following determination to intervene, the sequence advances to operation 222, which involves verifying that the contact sensor detects skin contact. Operation 222 comprises the processor querying the contact sensor positioned in the device structure that will be discussed in subsequent figures to confirm the wearable therapeutic unit maintains proper placement against the skin surface before enabling injection mechanism actuation. The contact verification at operation 222 prevents inappropriate injection attempts when the device has become dislodged from proper body placement, ensuring the injection mechanism 100 detailed in FIG. 1 delivers the therapeutic agent into tissue rather than discharging into air or clothing. Operation 222 functions by providing a hardware safety interlock that requires both detection logic confirmation and physical placement confirmation before permitting injection, implementing a multi-condition safety architecture that prevents autonomous injection during device handling, storage, or accidental triggering.

The sequence then advances to operation 224, which involves activating the injection mechanism to deliver the therapeutic agent. Operation 224 comprises the processor sending control signals to the mechanical actuator that will be discussed in subsequent figures, triggering release of the spring 110 described in FIG. 1 to drive the plunger 112 and expel the therapeutic agent 104 through the needle 106 into muscle tissue. In some embodiments, the activation at operation 224 executes the complete injection sequence including valve rupture, therapeutic agent expulsion, and needle deployment within timeframes measured in milliseconds to seconds. Operation 224 functions by converting the detection decision into physical therapeutic intervention, delivering intramuscular epinephrine that counteracts anaphylaxis through vasoconstriction reversing cardiovascular shock, bronchodilation relieving respiratory obstruction, and histamine receptor antagonism reducing continued allergic cascade progression.

Following injection activation, the sequence concludes at operation 226, which involves logging the injection event and transmitting an alert to an external device. Operation 226 comprises the processor recording event data including timestamp, physiological parameters at injection, confidence scores, and injection confirmation to memory while simultaneously transmitting alerts via the wireless communication module to the user's smartphone, emergency contacts, or medical monitoring services. The logging at operation 226 creates documentation enabling subsequent medical review of event characteristics and device performance. The alert transmission at operation 226 notifies caregivers or emergency services of anaphylaxis occurrence, enabling appropriate follow-up care including emergency department evaluation, replacement device provision, or allergen exposure investigation. Operation 226 functions by ensuring anaphylaxis events receive appropriate medical response beyond immediate epinephrine administration, addressing the comprehensive care requirements for severe allergic reactions that may include multi-phase responses requiring additional interventions or monitoring.

In some embodiments, the processor comprises a microcontroller with integrated analog-to-digital converters, digital signal processing capabilities, and wireless communication interfaces to minimize component count and power consumption. In some embodiments, the processor comprises a system-on-chip integrating central processing unit, memory, sensor interfaces, and radio frequency transceivers on a single integrated circuit. In some embodiments, the processor operates at clock frequencies ranging from 16 megahertz to 200 megahertz selected to balance processing performance against battery life requirements. In some embodiments, the processor implements power management modes including active processing during detection logic execution, low-power monitoring during baseline surveillance, and ultra-low-power sleep during inactive periods. In some embodiments, the processor executes firmware stored in read-only memory or flash memory, with firmware updates delivered wirelessly through the wireless communication module to enable computational routine improvements or parameter adjustments without hardware replacement.

In some embodiments, the baseline establishment at operation 208 comprises calculating rolling baseline values that update periodically during extended wear to track gradual physiological changes over days or weeks. In some embodiments, the baseline calculation excludes measurements during detected exercise periods, sleep periods, or other contexts where temporary physiological changes should not influence baseline characterization. In some embodiments, the baseline establishment incorporates time-of-day variations, calculating separate baseline values for morning, afternoon, evening, and nighttime periods to account for circadian rhythm effects on heart rate, body temperature, and autonomic tone. In some embodiments, the baseline values stored at operation 210 include confidence metrics or validity indicators that enable the processor to assess baseline reliability and determine whether recalibration is required due to insufficient initial data or drift over extended wear periods.

In some embodiments, the continuous monitoring at operation 204 samples respiratory sensors at rates ranging from 1 hertz to 100 hertz to capture breathing dynamics and acoustic features including wheeze frequencies and cough transients. In some embodiments, the cardiovascular sensor sampling at operation 204 occurs at rates ranging from 50 hertz to 500 hertz to resolve individual heartbeats and calculate heart rate variability metrics requiring precise inter-beat interval measurement. In some embodiments, the autonomic sensor sampling occurs at rates ranging from 0.1 hertz to 10 hertz sufficient for skin conductance and temperature dynamics that change more slowly than cardiovascular or respiratory parameters. In some embodiments, the processor implements adaptive sampling rates that increase during detected deviation periods to capture transient dynamics and decrease during stable periods to conserve power and extend battery life. In some embodiments, the processor applies digital filtering to sensor data including low-pass filtering to remove high-frequency noise, high-pass filtering to remove baseline drift, or band-pass filtering to isolate physiological signal frequencies while rejecting motion artifacts and environmental interference.

In some embodiments, the alert transmission at operation 226 comprises sending wireless messages via Bluetooth Low Energy to paired smartphones within communication range, via Wi-Fi to local network devices, or via cellular communication to remote monitoring services through integrated cellular modems. In some embodiments, the external device receiving alerts comprises dedicated emergency response applications configured to parse physiological data and initiate emergency service contact. In some embodiments, the external device comprises caregiver notification systems that alert family members or medical professionals through push notifications or text messages. In some embodiments, the external device comprises electronic health record interfaces that automatically document anaphylaxis events in patient medical records. In some embodiments, the alert messages include physiological data plots showing sensor trends preceding injection, confidence score histories demonstrating detection evolution, or device status information indicating battery level and sensor functionality to support medical evaluation. In some embodiments, the processor transmits periodic status updates confirming device operation and battery level to external devices during normal monitoring, enabling users to verify device functionality without intervention events and receive low-battery warnings prompting recharging or device replacement.

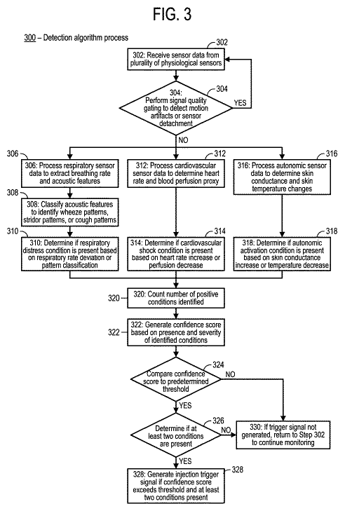

FIG. 3 illustrates a flowchart diagram of the multi-system anaphylaxis detection method 300 for the wearable autonomous anaphylaxis treatment device showing the analytical sequence that identifies anaphylaxis through concurrent physiological changes across multiple organ systems according to the embodiment described in FIG. 1. The detection method 300 represents the computational logic executed by the processor to distinguish true anaphylactic episodes from false positive conditions including anxiety reactions, exercise-induced changes, or isolated physiological variations.

The method 300 begins at operation 302, which involves receiving sensor data from the plurality of physiological sensors. Operation 302 comprises the processor obtaining digitized measurements from the respiratory sensor, cardiovascular sensor, and autonomic sensor as generated during the continuous monitoring described in FIG. 2. The sensor data received at operation 302 includes time-series measurements spanning recent monitoring intervals, providing the raw physiological information required for pattern analysis. Operation 302 functions by establishing the data input pathway that feeds subsequent detection logic, ensuring current physiological measurements are available for quality assessment and condition evaluation.

The method advances to decision point 304, which performs signal quality gating to detect motion artifacts or sensor detachment. Decision point 304 comprises the processor evaluating signal characteristics including amplitude ranges, noise levels, signal-to-noise ratios, or temporal consistency to identify corrupted measurements that could generate false detections. In some embodiments, the signal quality assessment examines whether sensor values fall within physiologically plausible ranges, detecting sensor detachment through impedance measurements or signal dropout patterns. In some embodiments, the quality gating analyzes motion artifact signatures from accelerometer data, identifying movement patterns that corrupt physiological measurements. When decision point 304 identifies poor signal quality, the method returns to operation 302 via the feedback pathway to await improved measurements, preventing detection decisions based on unreliable data. When decision point 304 confirms acceptable signal quality, the method proceeds to parallel processing of respiratory, cardiovascular, and autonomic data streams. Decision point 304 functions by filtering artifact-corrupted measurements that could trigger false positives, improving detection specificity while maintaining sensitivity for true anaphylaxis events occurring during physical activity or movement.

Following quality confirmation, the method executes three parallel analysis pathways. The respiratory analysis begins at operation 306, which involves processing respiratory sensor data to extract breathing rate and acoustic features. Operation 306 comprises the processor analyzing respiratory waveforms to identify inspiration and expiration cycles through zero-crossing detection or peak identification, calculating breathing rate in breaths per minute. The processor during operation 306 applies frequency-domain analysis through fast Fourier transform computation to respiratory acoustic signals, extracting spectral features characteristic of wheeze sounds typically occurring in frequency ranges from 400 hertz to 1600 hertz, stridor sounds at lower frequencies from 200 hertz to 600 hertz, or cough transients with broadband frequency content. Operation 306 functions by converting raw respiratory measurements into quantitative features suitable for pattern classification, isolating acoustic signatures that indicate airway obstruction or bronchospasm from normal breathing sounds.

The method continues to operation 308, which involves classifying the acoustic features to identify wheeze patterns, stridor patterns, or cough patterns. Operation 308 comprises the processor comparing extracted acoustic features against reference patterns representing pathological respiratory sounds. In some embodiments, the classification applies threshold comparisons where spectral power in wheeze-characteristic frequency bands exceeding predetermined levels indicates wheeze presence. In some embodiments, the classification implements pattern matching routines comparing temporal and spectral characteristics of detected sounds against stored wheeze, stridor, or cough templates. In some embodiments, the classification employs machine learning classifiers trained on labeled respiratory sound datasets to distinguish pathological sounds from normal breathing, throat clearing, or speech. Operation 308 functions by identifying specific respiratory sound abnormalities that indicate allergic airway response, distinguishing anaphylaxis-associated wheeze from other respiratory sounds through acoustic signature analysis.

The respiratory analysis concludes at operation 310, which involves determining whether the respiratory distress condition is present based on respiratory rate deviation or pattern classification results. Operation 310 comprises the processor evaluating multiple respiratory criteria including breathing rate increases relative to baseline values, irregular breathing patterns indicative of bronchospasm or labored respiration, or classified wheeze, stridor, or cough patterns exceeding confidence thresholds. In some embodiments, operation 310 identifies respiratory distress when breathing rate increases above baseline by percentage thresholds ranging from 20 percent to 50 percent sustained over time windows ranging from 15 seconds to 60 seconds. In some embodiments, operation 310 detects irregular patterns through breath-to-breath variability analysis or duty cycle calculations indicating prolonged expiration characteristic of airway obstruction. In some embodiments, operation 310 requires classifier confidence scores exceeding threshold values ranging from 0.6 to 0.9 on normalized confidence scales for wheeze or stridor detection. Operation 310 functions by integrating multiple respiratory indicators into a binary respiratory distress determination, enabling anaphylaxis detection through respiratory symptoms while reducing false positives from isolated breathing rate variations during normal activities.

Parallel to respiratory analysis, the cardiovascular analysis begins at operation 312, which involves processing cardiovascular sensor data to determine heart rate and blood perfusion proxy. Operation 312 comprises the processor analyzing photoplethysmography or electrocardiogram waveforms to identify cardiac cycles through peak detection routines, calculating heart rate from inter-beat intervals. The processor during operation 312 derives blood perfusion proxies from photoplethysmography signal amplitudes, where amplitude reductions indicate peripheral vasoconstriction and reduced tissue perfusion characteristic of cardiovascular shock. Operation 312 functions by extracting cardiovascular parameters that indicate both sympathetic activation through heart rate increases and circulatory compromise through perfusion reductions, capturing the dual cardiovascular responses occurring during anaphylaxis.

The cardiovascular analysis concludes at operation 314, which involves determining whether the cardiovascular shock condition is present based on heart rate increase or perfusion decrease. Operation 314 comprises the processor evaluating cardiovascular criteria including heart rate elevations relative to baseline values, heart rate variability decreases indicating reduced autonomic balance and sympathetic dominance, blood perfusion reductions indicating shock, or temperature decreases indicating peripheral vasoconstriction. In some embodiments, operation 314 identifies cardiovascular shock when heart rate increases above baseline by percentage thresholds ranging from 25 percent to 40 percent persisting over time windows ranging from 20 seconds to 90 seconds. In some embodiments, operation 314 detects perfusion compromise when photoplethysmography amplitude decreases below baseline by percentage thresholds ranging from 15 percent to 35 percent. In some embodiments, operation 314 identifies peripheral vasoconstriction when skin temperature decreases below baseline by temperature thresholds ranging from 1 degree Celsius to 3 degrees Celsius indicating reduced peripheral blood flow. Operation 314 functions by recognizing cardiovascular manifestations of anaphylaxis through multiple indicators, distinguishing anaphylaxis-associated cardiovascular shock from exercise-induced heart rate elevation through concurrent perfusion or temperature changes.

Parallel to respiratory and cardiovascular analyses, the autonomic analysis begins at operation 316, which involves processing autonomic sensor data to determine skin conductance and skin temperature changes. Operation 316 comprises the processor analyzing galvanic skin response measurements to quantify skin conductance changes indicating sympathetic nervous system activation and sweat gland activity, and analyzing thermistor measurements to track skin temperature variations reflecting autonomic thermoregulation and peripheral circulation. Operation 316 functions by extracting autonomic parameters that indicate stress responses and circulatory changes accompanying anaphylaxis, providing additional physiological evidence beyond respiratory and cardiovascular systems.

The autonomic analysis concludes at operation 318, which involves determining whether the autonomic activation condition is present based on skin conductance increase or temperature decrease. Operation 318 comprises the processor evaluating autonomic criteria including skin conductance elevations indicating sympathetic arousal or skin temperature reductions indicating peripheral vasoconstriction. In some embodiments, operation 318 identifies autonomic activation when skin conductance increases above baseline by percentage thresholds ranging from 30 percent to 60 percent sustained over time windows ranging from 10 seconds to 45 seconds. In some embodiments, operation 318 detects peripheral vasoconstriction through temperature criteria previously described. Operation 318 functions by recognizing autonomic nervous system responses to anaphylaxis, supplementing respiratory and cardiovascular evidence with additional physiological confirmation.

Following completion of the three parallel analyses, the method advances to operation 320, which involves counting the number of positive conditions identified from respiratory distress, cardiovascular shock, and autonomic activation determinations. Operation 320 comprises the processor tallying how many of the three physiological conditions were identified as present, generating a count value ranging from zero to three. Operation 320 functions by quantifying multi-system involvement, providing the foundation for the multi-condition requirement that distinguishes anaphylaxis affecting multiple organ systems from isolated physiological changes affecting single systems.

The method continues to operation 322, which involves generating the confidence score based on presence and severity of identified conditions. Operation 322 comprises the processor calculating a numerical confidence value indicating detection certainty through weighted combination of condition presence indicators and deviation magnitudes. In some embodiments, the confidence score calculation applies equal weighting to respiratory, cardiovascular, and autonomic conditions. In some embodiments, the confidence score applies differential weighting that prioritizes respiratory and cardiovascular evidence over autonomic evidence based on clinical significance. In some embodiments, the confidence score incorporates deviation severity where larger deviations from baseline contribute higher confidence values than marginal deviations. In some embodiments, the confidence score ranges from 0.0 to 1.0 on normalized scales or from 0 to 100 on percentage scales. Operation 322 functions by converting multi-parameter physiological evidence into a single decision metric, enabling threshold-based determination of whether detection confidence justifies autonomous intervention.

The method then reaches decision point 324, which compares the confidence score to the predetermined threshold. Decision point 324 comprises the processor evaluating whether the confidence score generated at operation 322 exceeds a threshold value calibrated to balance early detection sensitivity against false positive specificity. In some embodiments, the confidence threshold ranges from 0.65 to 0.85 on normalized confidence scales, with lower thresholds favoring sensitivity and higher thresholds favoring specificity. When decision point 324 determines the confidence score remains below threshold, the method advances to operation 330 to return to continuous monitoring without triggering intervention. When decision point 324 determines the confidence score exceeds threshold, the method advances to decision point 326 for multi-condition verification. Decision point 324 functions by implementing the confidence requirement that ensures detection certainty justifies intervention, preventing therapeutic delivery for ambiguous physiological patterns while enabling rapid response for clear anaphylaxis presentations.

Following confidence confirmation, the method reaches decision point 326, which determines whether at least two conditions are present from respiratory distress, cardiovascular shock, and autonomic activation. Decision point 326 comprises the processor evaluating the condition count from operation 320 to verify multi-system involvement. When decision point 326 determines fewer than two conditions are present, indicating single-system changes insufficient for anaphylaxis diagnosis, the method advances to operation 330 to return to monitoring. When decision point 326 determines two or more conditions are present, indicating multi-system involvement consistent with anaphylaxis, the method advances to operation 328 to generate the injection trigger signal. Decision point 326 functions by implementing the multi-system requirement that distinguishes anaphylaxis from false positive conditions, preventing intervention for anxiety-induced tachycardia without respiratory involvement, exercise-induced cardiovascular changes with coordinated respiratory response, or isolated autonomic responses to environmental temperature.

Following multi-condition confirmation, the method advances to operation 328, which involves generating the injection trigger signal when confidence score exceeds threshold and at least two conditions are present. Operation 328 comprises the processor setting a trigger flag or generating a control signal that initiates the safety verification sequence described in a subsequent figure. The trigger signal generated at operation 328 represents the algorithmic determination that anaphylaxis is occurring with sufficient confidence to justify autonomous intervention. Operation 328 functions by converting the multi-parameter physiological analysis into a binary intervention decision, advancing the operational sequence from detection to therapeutic delivery.

Alternatively, when either confidence threshold or multi-condition requirements are not satisfied, the method reaches operation 330, which returns to operation 302 to continue monitoring. Operation 330 comprises the processor maintaining surveillance without intervention, enabling ongoing physiological assessment that may detect anaphylaxis development in subsequent monitoring cycles. Operation 330 functions by preserving continuous monitoring throughout periods when detection criteria are not met, ensuring the system remains vigilant for anaphylaxis onset without generating false positive interventions.

In some embodiments, operation 314 detects reduced heart rate variability when inter-beat interval standard deviation decreases below baseline by percentage thresholds ranging from 20 percent to 50 percent, indicating sympathetic nervous system dominance characteristic of cardiovascular stress.

In some embodiments, the respiratory rate deviation threshold comprises percentage increases from baseline ranging from 15 percent to 60 percent depending on desired sensitivity and user population characteristics. In some embodiments, the time window for sustained respiratory rate elevation ranges from 10 seconds to 120 seconds, with shorter windows enabling faster detection and longer windows reducing transient artifact sensitivity. In some embodiments, the wheeze classification threshold comprises spectral power thresholds ranging from 20 decibels to 40 decibels above background noise levels in characteristic frequency bands. In some embodiments, the heart rate deviation threshold comprises percentage increases from baseline ranging from 20 percent to 50 percent for adult populations or 30 percent to 60 percent for pediatric populations reflecting higher baseline variability. In some embodiments, the perfusion decrease threshold comprises amplitude reductions ranging from 10 percent to 40 percent below baseline photoplethysmography values. In some embodiments, the skin conductance increase threshold comprises percentage elevations from baseline ranging from 25 percent to 70 percent depending on baseline autonomic tone variability.

In some embodiments, the signal quality gating at decision point 304 applies accelerometer-based motion detection, excluding data during movement exceeding acceleration thresholds ranging from 0.5 g to 2 g indicating physical activity. In some embodiments, the signal quality assessment evaluates signal-to-noise ratios requiring minimum values ranging from 10 decibels to 30 decibels for acceptable signal quality. In some embodiments, the quality gating implements impedance monitoring for electrocardiogram or galvanic skin response sensors, detecting sensor detachment through impedance increases exceeding threshold values ranging from 10 kilohms to 100 kilohms. In some embodiments, the quality assessment analyzes temporal signal characteristics including missing sample rates or signal dropout durations, rejecting data with dropout periods exceeding thresholds ranging from 0.5 seconds to 2 seconds.

In some embodiments, the acoustic feature classification at operation 308 implements support vector machine classifiers trained on labeled respiratory sound databases. In some embodiments, the classification employs convolutional neural networks analyzing spectrogram representations of respiratory sounds. In some embodiments, the classification applies decision tree ensembles combining multiple acoustic features including spectral peaks, temporal patterns, and harmonic ratios. In some embodiments, the classification implements template matching comparing detected sounds against stored wheeze, stridor, and cough exemplars through correlation analysis. In some embodiments, the respiratory analysis incorporates breathing pattern irregularity detection through breath-to-breath interval standard deviation calculations, identifying irregularity when standard deviations exceed threshold values ranging from 15 percent to 35 percent of mean breathing intervals.

In some embodiments, the confidence score calculation at operation 322 implements linear weighted summation where respiratory condition contributes weights ranging from 0.35 to 0.45, cardiovascular condition contributes weights ranging from 0.35 to 0.45, and autonomic condition contributes weights ranging from 0.15 to 0.25. In some embodiments, the confidence calculation implements non-linear scoring where simultaneous presence of respiratory and cardiovascular conditions generates confidence scores exceeding simple additive combinations. In some embodiments, the confidence score incorporates temporal persistence factors where conditions sustained over longer durations contribute higher confidence than transient detections. In some embodiments, the confidence scoring adjusts thresholds based on user history, reducing thresholds for users with prior anaphylaxis episodes documented through previous interventions or increasing thresholds for users with frequent false positive patterns during initial wear periods.

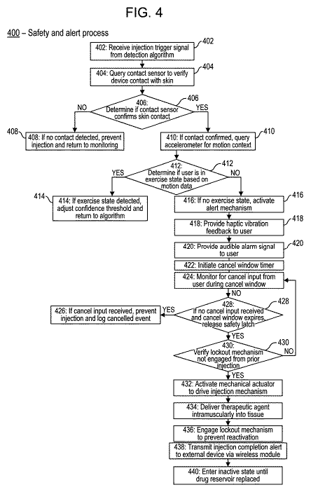

FIG. 4 illustrates a flowchart diagram of the safety interlock and alert sequence 400 for the wearable autonomous anaphylaxis treatment device showing the verification and notification operations that occur between anaphylaxis detection and therapeutic delivery according to the embodiment described in FIG. 1. The safety sequence 400 represents the multi-stage verification logic that prevents inappropriate injection through hardware interlocks, context-aware gating, and user notification mechanisms.

The sequence 400 begins at operation 402, which involves receiving the injection trigger signal from the detection method described in FIG. 3. Operation 402 comprises the processor obtaining the trigger signal generated when confidence scores and multi-condition requirements are satisfied, initiating the safety verification pathway. Operation 402 functions by transitioning from detection logic to intervention logic, establishing the entry point for safety assessment that precedes mechanical actuation.

The sequence advances to operation 404, which involves querying the contact sensor to verify device contact with skin. Operation 404 comprises the processor reading the contact sensor state to confirm the wearable therapeutic unit maintains proper body placement. The contact sensor generates signals through pressure-sensitive elements, capacitive sensing detecting skin proximity, or resistance measurements confirming conductive skin contact. Operation 404 functions by implementing the first hardware safety interlock that requires both algorithmic detection decision and physical placement confirmation before permitting injection.

The sequence reaches decision point 406, which determines whether the contact sensor confirms skin contact. Decision point 406 comprises the processor evaluating contact sensor signals against threshold criteria indicating reliable skin interface. When decision point 406 determines contact is not detected, the sequence advances to operation 408 to prevent injection and return to monitoring, ensuring the injection mechanism does not discharge when the device is dislodged or removed. When decision point 406 confirms contact, the sequence advances to operation 410 for motion context evaluation. Decision point 406 functions by preventing injection attempts during device handling, storage, or accidental dislodgement situations where therapeutic delivery would be inappropriate or ineffective.

Following contact confirmation, the sequence advances to operation 410, which involves querying the accelerometer for motion context. Operation 410 comprises the processor analyzing accelerometer data to identify movement patterns characteristic of exercise or vigorous physical activity that could generate physiological changes mimicking anaphylaxis. Operation 410 functions by providing context-aware gating that distinguishes exercise-induced physiological responses from anaphylaxis, reducing false positive interventions during normal activities.

The sequence reaches decision point 412, which determines whether the user is in an exercise state based on motion data. Decision point 412 comprises the processor evaluating accelerometer patterns against movement signatures indicating sustained physical activity. In some embodiments, decision point 412 identifies exercise through acceleration magnitudes exceeding threshold values ranging from 0.5 g to 2 g sustained over periods ranging from 30 seconds to 120 seconds. In some embodiments, the exercise detection analyzes movement frequency content characteristic of walking, running, or cycling through spectral analysis. When decision point 412 identifies exercise state, the sequence advances to operation 414 to adjust confidence thresholds and return to detection logic, requiring higher evidence levels for intervention during contexts where elevated heart rate and respiratory rate represent normal physiological responses. When decision point 412 determines no exercise state, the sequence advances to operation 416 to initiate user alerting. Decision point 412 functions by implementing context-aware sensitivity adjustment that maintains detection capability during exercise while reducing false positive risks.

Following context verification, the sequence advances to operation 416, which involves activating the alert mechanism. Operation 416 comprises the processor energizing alert components to notify the user of impending intervention. Operation 416 functions by implementing the alert-before-inject strategy that provides conscious users opportunity to cancel intervention if detection is erroneous, balancing autonomous operation with user override capability.

The sequence continues to operation 418, which involves providing haptic vibration feedback to the user. Operation 418 comprises the processor activating vibration motors or haptic actuators that generate tactile sensations alerting the user to device activity. In some embodiments, operation 418 generates vibration patterns with durations ranging from 0.5 seconds to 3 seconds at frequencies ranging from 100 hertz to 300 hertz. Operation 418 functions by providing non-audible notification suitable for environments where audible alerts may be disruptive or unnoticed.

The sequence advances to operation 420, which involves providing audible alarm signal to the user. Operation 420 comprises the processor activating audio transducers that generate sound notifications. In some embodiments, operation 420 generates alarm tones with frequencies ranging from 1000 hertz to 4000 hertz at sound pressure levels ranging from 70 decibels to 90 decibels. Operation 420 functions by providing attention-capturing notification that alerts users and nearby individuals to imminent intervention.

Following alert activation, the sequence advances to operation 422, which involves initiating the cancel window timer. Operation 422 comprises the processor starting a countdown timer that defines the duration during which user cancellation input will be accepted. In some embodiments, the cancel window duration ranges from 3 seconds to 15 seconds, balancing adequate response time for conscious users against intervention delay for unconscious users. Operation 422 functions by establishing the temporal window for human-in-the-loop override, enabling autonomous operation to proceed unless user intervention prevents injection.

The sequence continues to operation 424, which involves monitoring for cancel input from the user during the cancel window. Operation 424 comprises the processor continuously checking user input interfaces including buttons, touchscreens, or smartphone application controls for cancellation commands. Operation 424 functions by providing the monitoring mechanism that enables user override, capturing manual intervention attempts during the alert period.

The sequence reaches decision point 426, which evaluates whether cancel input is received. Decision point 426 comprises the processor determining whether user cancellation commands were detected during the cancel window. When decision point 426 detects cancel input, the sequence advances to operation 426 to prevent injection and log the cancelled event, respecting user judgment that intervention is inappropriate. When decision point 426 determines no cancel input occurred, the sequence advances to decision point 428 to evaluate timer expiration. Decision point 426 functions by implementing the user override mechanism that enables conscious users to prevent therapeutic delivery based on symptom assessment or context knowledge.

The sequence reaches decision point 428, which determines whether the cancel window has expired. Decision point 428 comprises the processor evaluating whether the timer initiated at operation 422 has completed without receiving cancel input. When decision point 428 determines the cancel window remains active, the sequence returns to operation 424 to continue monitoring for cancel input. When decision point 428 determines the window has expired without cancellation, the sequence advances to operation 430 for lockout verification, indicating user acceptance or inability to respond justifying autonomous intervention. Decision point 428 functions by transitioning from alert phase to actuation phase after providing adequate cancellation opportunity.

Following cancel window expiration, the sequence reaches decision point 430, which verifies the lockout mechanism is not engaged from prior injection. Decision point 430 comprises the processor querying lockout status to confirm the device contains active therapeutic agent and has not previously delivered a dose. When decision point 430 determines lockout is engaged, the sequence advances to operation 440 to enter inactive state, preventing multiple injections from a single-dose device. When decision point 430 confirms lockout is not engaged, the sequence advances to operation 432 to activate the injection mechanism. Decision point 430 functions by implementing the single-dose safety feature that prevents repeated actuation after therapeutic delivery.

Following lockout verification, the sequence advances to operation 432, which involves activating the mechanical actuator to drive the injection mechanism. Operation 432 comprises the processor sending control signals that release the spring 110 described in FIG. 1, initiating the mechanical injection sequence. Operation 432 functions by converting the multi-stage safety verification into physical actuation command, triggering therapeutic delivery after confirming detection confidence, device placement, motion context, user non-objection, and single-use status.

The sequence continues to operation 434, which involves delivering the therapeutic agent intramuscularly into tissue. Operation 434 comprises the mechanical injection sequence where the spring 110 drives the plunger 112 to expel the therapeutic agent 104 through the needle 106 as described in FIG. 1. Operation 434 functions by accomplishing the therapeutic objective of autonomous anaphylaxis intervention, delivering intramuscular epinephrine that counteracts allergic cascade progression.

Following therapeutic delivery, the sequence advances to operation 436, which involves engaging the lockout mechanism to prevent reactivation. Operation 436 comprises mechanical or electronic lockout implementation that prevents subsequent actuation attempts. In some embodiments, operation 436 engages mechanical latches that prevent spring recompression. In some embodiments, operation 436 sets electronic flags in non-volatile memory that prevent processor-initiated actuation commands. Operation 436 functions by implementing the single-dose enforcement that ensures devices deliver only one therapeutic dose before requiring replacement.

The sequence continues to operation 438, which involves transmitting injection completion alert to external devices via wireless module. Operation 438 comprises the processor sending notification messages including event timestamp, pre-injection physiological data, confidence scores, and device identification through wireless communication. In some embodiments, operation 438 transmits alerts via Bluetooth Low Energy to paired smartphones, via Wi-Fi to local networks, or via cellular communication to emergency services. Operation 438 functions by notifying caregivers, family members, or medical professionals of anaphylaxis occurrence, enabling appropriate follow-up including emergency department transport, allergen identification, or device replacement.

The sequence concludes at operation 440, which involves entering inactive state until drug reservoir is replaced. Operation 440 comprises the processor transitioning to a post-intervention mode where monitoring continues but actuation is disabled. Operation 440 functions by maintaining device awareness of intervention status while preventing inadvertent reactivation, prompting users to obtain replacement devices.

In some embodiments, the contact sensor comprises capacitive sensing elements detecting skin proximity through electrical field changes. In some embodiments, the contact sensor comprises resistive pressure sensors responding to device compression against skin. In some embodiments, the contact sensor comprises impedance measurement circuits detecting low-impedance paths through skin contact.

In some embodiments, the haptic feedback at operation 418 implements escalating vibration patterns with increasing intensity to capture attention. In some embodiments, the audible alarm at operation 420 generates distinctive multi-tone sequences differentiating anaphylaxis alerts from other notifications. In some embodiments, the alert mechanisms coordinate with smartphone applications displaying visual alerts showing confidence scores and detected symptoms.

In some embodiments, the cancel input mechanism comprises physical buttons on the device requiring sustained pressure to prevent accidental cancellation. In some embodiments, the cancel mechanism comprises smartphone application controls requiring authentication to prevent unintended override. In some embodiments, the cancel window duration adapts based on user responsiveness history, extending for users with documented slower response times.

In some embodiments, the lockout mechanism at operation 436 comprises mechanical ratchets preventing spring recompression. In some embodiments, the lockout comprises electronic fuses permanently disabled after actuation. In some embodiments, the lockout status transmits periodic alerts reminding users of inactive device state requiring replacement.

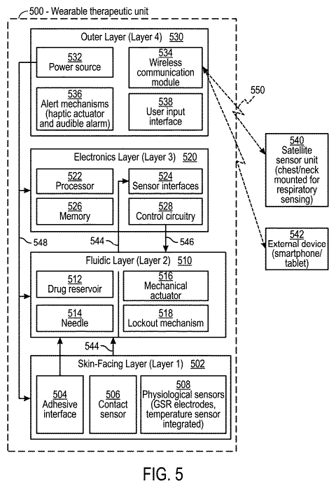

FIG. 5 illustrates a block diagram of the layered system architecture 500 for the wearable autonomous anaphylaxis treatment device showing the spatial arrangement and interconnection of functional layers according to the embodiment described in FIG. 1. The wearable therapeutic unit 500 represents the integrated physical structure comprising four distinct layers that collectively provide sensing, processing, actuation, and power management capabilities.

The wearable therapeutic unit 500 comprises a skin-facing layer 502 positioned for direct body contact. The skin-facing layer 502 includes the adhesive interface 504 configured to secure the unit 500 to skin through biocompatible adhesive materials, the contact sensor 506 configured to verify skin contact as described in FIG. 4, and the physiological sensors 508 comprising the integrated galvanic skin response electrodes and temperature sensor described in previous figures. The skin-facing layer 502 connects to the fluidic layer 510 positioned above through structural supports and electrical pathways enabling sensor signal transmission to the electronics layer 520. The skin-facing layer 502 functions by establishing the user interface that maintains device placement while providing physiological measurement access, creating the foundation for continuous monitoring and reliable therapeutic delivery.

The fluidic layer 510 positioned above the skin-facing layer 502 comprises the drug reservoir 512 containing therapeutic agent as described in FIG. 1, the needle 514 providing the fluid pathway for intramuscular delivery, the mechanical actuator 516 configured to drive injection as described in FIGS. 1 and 4, and the lockout mechanism 518 preventing reactivation as described in FIG. 4. The fluidic layer 510 connects to the electronics layer 520 through control signal pathways shown as connection 546 enabling the processor 522 to trigger actuation. The fluidic layer 510 functions by integrating therapeutic storage and delivery mechanisms in a dedicated compartment that isolates drug-contact surfaces from electronic components, enabling sterile fluid pathway maintenance while supporting mechanical actuation.

The electronics layer 520 positioned above the fluidic layer 510 comprises the processor 522 executing the detection and safety logic described in FIGS. 2, 3, and 4, the sensor interfaces 524 providing analog-to-digital conversion and signal conditioning for physiological measurements, the memory 526 storing baseline values and firmware as described in FIG. 2, and the control circuitry 528 providing actuation driver electronics. The electronics layer 520 receives sensor data through connection 544 from the skin-facing layer 502, sends control signals through connection 546 to the fluidic layer 510, and receives power through connection 548 from the outer layer 530. The electronics layer 520 functions by providing the computational core that transforms sensor inputs into therapeutic decisions, executing the autonomous detection and intervention logic while coordinating communication and power management functions.

The outer layer 530 positioned above the electronics layer 520 comprises the power source 532 providing electrical energy for system operation, the wireless communication module 534 enabling alert transmission as described in FIG. 4, the alert mechanisms 536 including haptic actuator and audible alarm described in FIG. 4, and the user input interface 538 enabling cancel input described in FIG. 4. The outer layer 530 distributes power to the electronics layer 520, skin-facing layer 502 sensors, and fluidic layer 510 actuator through connection 548. The outer layer 530 communicates wirelessly through connection 550 to the external device 542 including smartphones or medical monitoring systems. The outer layer 530 functions by providing power, communication, and user interface capabilities that support system operation while remaining accessible from the device exterior for user interaction and wireless transmission.

The wearable therapeutic unit 500 may include the satellite sensor unit 540 positioned remotely from the main unit 500 for enhanced respiratory monitoring. The satellite sensor unit 540 comprises acoustic sensors positioned on chest or neck for improved respiratory sound detection. The satellite sensor unit 540 communicates with the electronics layer 520 through wireless connection 550 enabling distributed sensor architectures. In some embodiments, the satellite sensor unit 540 comprises a separate wearable module with independent power and wireless transmission. In some embodiments, the satellite sensor unit 540 integrates respiratory and cardiovascular sensors in a chest-mounted configuration while the main unit 500 positions on the thigh for intramuscular delivery access.

The external device 542 comprises smartphones, tablets, or medical monitoring systems receiving alerts through wireless connection 550. The external device 542 executes applications displaying physiological data, confidence scores, and intervention notifications. The external device 542 provides user interfaces for cancel input, device configuration, or emergency service contact.

In some embodiments, the adhesive interface 504 comprises medical-grade silicone adhesives maintaining skin attachment over wear periods ranging from 12 hours to 72 hours. In some embodiments, the skin-facing layer 502 comprises flexible polymer substrates conforming to body contours. In some embodiments, the physiological sensors 508 integrate electrocardiogram electrodes, photoplethysmography sensors, or additional strain sensors for swelling detection.

In some embodiments, the physiological sensors 508 integrate strain sensors configured to detect tissue swelling through mechanical deformation measurement indicating angioedema development.

In some embodiments, the fluidic layer 510 incorporates thermal insulation materials maintaining drug reservoir 512 temperature within specified ranges. In some embodiments, the mechanical actuator 516 comprises spring assemblies generating forces ranging from 25 Newtons to 45 Newtons. In some embodiments, the lockout mechanism 518 comprises mechanical latches, electronic fuses, or irreversible mechanical deformation preventing reuse.

In some embodiments, the processor 522 comprises system-on-chip implementations operating at clock frequencies ranging from 16 megahertz to 200 megahertz. In some embodiments, the memory 526 comprises flash memory capacities ranging from 256 kilobytes to 2 megabytes. In some embodiments, the sensor interfaces 524 comprise multi-channel analog-to-digital converters with resolutions ranging from 12 bits to 16 bits.

In some embodiments, the power source 532 comprises thin-film lithium-ion batteries with capacities ranging from 100 milliampere-hours to 500 milliampere-hours supporting operational periods ranging from 24 hours to 168 hours. In some embodiments, the outer layer 530 includes solar cells providing supplemental charging. In some embodiments, the wireless communication module 534 supports Bluetooth Low Energy, Wi-Fi, or cellular protocols with transmission ranges from 10 meters to 100 meters for Bluetooth or unlimited range for cellular implementations.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

I/We claim:

1. A wearable autonomous anaphylaxis treatment device comprising:
a plurality of physiological sensors configured to continuously monitor a user and generate sensor data, wherein the plurality of physiological sensors comprises a respiratory sensor configured to detect at least one of breathing rate, breathing pattern, wheeze, stridor, or cough, a cardiovascular sensor configured to detect at least one of heart rate, heart rate variability, or blood perfusion, and an autonomic sensor configured to detect at least one of skin conductance or skin temperature;
a processor in communication with the plurality of physiological sensors, wherein the processor is configured to receive the sensor data from the plurality of physiological sensors, establish a baseline physiological state for the user based on the sensor data, detect deviations from the baseline physiological state, identify a respiratory distress condition based on data from the respiratory sensor, identify a cardiovascular shock condition based on data from the cardiovascular sensor, generate a confidence score based on presence of the respiratory distress condition and the cardiovascular shock condition, and generate an injection trigger signal when the confidence score exceeds a threshold and at least two of the respiratory distress condition, the cardiovascular shock condition, or an autonomic activation condition are present;
a drug reservoir containing a therapeutic agent;
an injection mechanism in fluid communication with the drug reservoir and in communication with the processor, wherein the injection mechanism is configured to deliver an intramuscular injection in response to the injection trigger signal to deliver the therapeutic agent from the drug reservoir into tissue of the user; and
a contact sensor positioned to detect contact with skin of the user, wherein the injection mechanism is configured to deliver the therapeutic agent when both the injection trigger signal is generated and the contact sensor detects contact with the skin of the user.

2. The wearable autonomous anaphylaxis treatment device of claim 1, wherein the therapeutic agent comprises epinephrine.

3. The wearable autonomous anaphylaxis treatment device of claim 2, wherein the drug reservoir comprises a sealed single-use cartridge, wherein the injection mechanism comprises a valve in fluid communication with the drug reservoir, wherein the valve is configured to rupture in response to the injection trigger signal to enable delivery of the epinephrine from the drug reservoir.

4. The wearable autonomous anaphylaxis treatment device of claim 3, wherein the injection mechanism comprises a spring-actuated needle, a plunger operatively connected to the spring-actuated needle, and a spring configured to drive the plunger to expel the epinephrine from the drug reservoir through the spring-actuated needle, wherein the spring is configured to generate sufficient force to deliver an intramuscular injection.

5. The wearable autonomous anaphylaxis treatment device of claim 1, wherein the respiratory sensor comprises an acoustic sensor configured to detect audible respiratory sounds, wherein the cardiovascular sensor comprises at least one of a photoplethysmography sensor or an electrocardiogram sensor, wherein the autonomic sensor comprises a galvanic skin response sensor configured to measure skin conductance.

6. The wearable autonomous anaphylaxis treatment device of claim 5, wherein the processor is further configured to extract features from the audible respiratory sounds, classify the features to identify at least one of wheeze patterns, stridor patterns, or cough patterns, and identify the respiratory distress condition when the at least one of wheeze patterns, stridor patterns, or cough patterns exceeds a classifier threshold.

7. The wearable autonomous anaphylaxis treatment device of claim 1, wherein the processor is configured to establish the baseline physiological state by monitoring the sensor data over a calibration period, calculating baseline values for at least heart rate, respiratory rate, skin conductance, and skin temperature, and storing the baseline values, wherein the processor is configured to detect deviations from the baseline physiological state by comparing real-time sensor data to the baseline values.

8. The wearable autonomous anaphylaxis treatment device of claim 7, wherein the processor is configured to identify the respiratory distress condition when at least one of a respiratory rate increases above a baseline respiratory rate by a first percentage threshold, the respiratory rate exhibits an irregular pattern indicative of bronchospasm, or the wheeze patterns exceed the classifier threshold.

9. The wearable autonomous anaphylaxis treatment device of claim 7, wherein the processor is configured to identify the cardiovascular shock condition when at least one of a heart rate increases above a baseline heart rate by a second percentage threshold and persists for a time window, a blood perfusion proxy measured by the cardiovascular sensor decreases below a baseline perfusion value by a third percentage threshold, or the skin temperature decreases indicating peripheral vasoconstriction.

10. The wearable autonomous anaphylaxis treatment device of claim 1, further comprising an alert mechanism in communication with the processor, wherein the processor is configured to activate the alert mechanism to provide a warning signal to the user after generation of the injection trigger signal and before activation of the injection mechanism, wherein the processor is configured to receive a cancel input from the user during a cancel window following activation of the alert mechanism, wherein the processor is configured to prevent activation of the injection mechanism when the cancel input is received during the cancel window.

11. The wearable autonomous anaphylaxis treatment device of claim 10, wherein the alert mechanism comprises at least one of a haptic actuator configured to provide vibration feedback or an audible alarm configured to emit an audible signal.

12. The wearable autonomous anaphylaxis treatment device of claim 1, further comprising an accelerometer configured to detect motion of the user, wherein the processor is in communication with the accelerometer and configured to receive motion data from the accelerometer, identify an exercise state based on the motion data, and adjust the threshold for the confidence score when the exercise state is identified to reduce likelihood of generating the injection trigger signal during exercise.

13. The wearable autonomous anaphylaxis treatment device of claim 1, wherein the processor is configured to identify the autonomic activation condition when at least one of the skin conductance measured by the autonomic sensor increases above a baseline skin conductance by a fourth percentage threshold or the skin temperature measured by the autonomic sensor decreases below a baseline skin temperature by a fifth percentage threshold.

14. A wearable autonomous anaphylaxis treatment system comprising:
a wearable therapeutic unit configured to be worn on a thigh of a user, wherein the wearable therapeutic unit comprises a layered structure having a skin-facing layer, a fluidic layer positioned above the skin-facing layer, an electronics layer positioned above the fluidic layer, and an outer layer positioned above the electronics layer, wherein the skin-facing layer comprises an adhesive interface configured to secure the wearable therapeutic unit to skin of the user and a contact sensor configured to detect contact with the skin, wherein the fluidic layer comprises a drug reservoir containing a therapeutic agent, a needle in fluid communication with the drug reservoir, and a mechanical actuator configured to drive the needle to deliver the therapeutic agent intramuscularly into tissue of the user, wherein the electronics layer comprises a processor and sensor interfaces in communication with the processor, wherein the outer layer comprises a power source and a wireless communication module;
a plurality of physiological sensors in communication with the processor through the sensor interfaces, wherein the plurality of physiological sensors is configured to generate sensor data indicative of physiological conditions of the user; and
wherein the processor is configured to analyze the sensor data to detect concurrent physiological changes across multiple physiological parameters indicative of anaphylaxis, generate a trigger signal when the concurrent physiological changes are detected, and activate the mechanical actuator in response to both the trigger signal and confirmation from the contact sensor that the wearable therapeutic unit is in contact with the skin to cause delivery of the therapeutic agent into the tissue of the user.

15. The wearable autonomous anaphylaxis treatment system of claim 14, wherein the therapeutic agent comprises epinephrine.

16. The wearable autonomous anaphylaxis treatment system of claim 14, wherein the plurality of physiological sensors comprises a respiratory sensor configured to detect at least one of breathing rate, breathing pattern, wheeze, stridor, or cough, a cardiovascular sensor configured to detect at least one of heart rate, heart rate variability, or blood perfusion, and an autonomic sensor configured to detect at least one of skin conductance or skin temperature, wherein the concurrent physiological changes comprise changes detected by at least two of the respiratory sensor, the cardiovascular sensor, or the autonomic sensor.

17. The wearable autonomous anaphylaxis treatment system of claim 14, wherein the fluidic layer further comprises a lockout mechanism configured to prevent reactivation of the mechanical actuator after delivery of the therapeutic agent, wherein the lockout mechanism is configured to maintain the wearable therapeutic unit in an inactive state until the drug reservoir is replaced.

18. The wearable autonomous anaphylaxis treatment system of claim 14, wherein the mechanical actuator comprises a microneedle array configured to penetrate skin of the user to deliver the therapeutic agent, wherein the microneedle array is configured to achieve intramuscular delivery through thermally expandable material positioned to drive the therapeutic agent from the drug reservoir through the microneedle array.

19. A method of autonomously treating anaphylaxis in a user wearing a wearable device, the method comprising:
a continuously monitoring a plurality of physiological parameters of the user using a plurality of physiological sensors of the wearable device to generate sensor data, wherein the plurality of physiological parameters comprises respiratory function, cardiovascular function, and autonomic function;
establishing a baseline physiological state for the user by analyzing the sensor data over a calibration period;
detecting deviations from the baseline physiological state by comparing real-time sensor data to the baseline physiological state;
identifying a respiratory distress condition when the sensor data from a respiratory sensor indicates at least one of an increased breathing rate, an irregular breathing pattern, wheeze, stridor, or cough above a baseline respiratory function;
identifying a cardiovascular shock condition when the sensor data from a cardiovascular sensor indicates at least one of an increased heart rate, decreased heart rate variability, or decreased blood perfusion relative to a baseline cardiovascular function;
generating a confidence score based on presence of at least two conditions selected from the respiratory distress condition, the cardiovascular shock condition, and an autonomic activation condition;
generating an injection trigger signal when the confidence score exceeds a predetermined threshold;
verifying contact between the wearable device and skin of the user using a contact sensor; and
delivering a therapeutic agent intramuscularly into tissue of the user using an injection mechanism of the wearable device in response to both the injection trigger signal and verification of contact between the wearable device and the skin of the user.

20. The method of claim 19, further comprising providing a warning signal to the user after generating the injection trigger signal and before delivering the therapeutic agent, receiving a cancel input from the user during a cancel window following the warning signal, and preventing delivery of the therapeutic agent when the cancel input is received during the cancel window.
